(19) United States
(12) Patent Application Publication
(10) Pub. No.: US 2026/0334849 A1
(43) Pub. Date: Oct. 29, 2026

(54) CORTICAL REVERBERATION MONITORING SYSTEM FOR NEUROLOGICAL ASSESSMENT

(71) Applicant: Regents of the University of Minnesota, Minneapolis, MN (US)

(72) Inventors: David Darrow, Minneapolis, MN (US); Alexander Herman, Minneapolis, MN (US); Seth König, Minneapolis, MN (US); Theoden Netoff, Minneapolis, MN (US); Samuel Cramer, Minneapolis, MN (US); Alik Widge, Minneapolis, MN (US)

(21) Appl. No.: 19/656,466

(22) Filed: Apr. 23, 2026

Related U.S. Application Data
(60) Provisional application No. 63/793,283, filed on Apr. 23, 2025.

(51) Int. Cl. A61B 5/383; A61B 5/00; A61B 5/293; A61B 5/37; A61B 5/372; A61B 5/03

(52) U.S. Cl. CPC A61B 5/383; A61B 5/293; A61B 5/37; A61B 5/372; A61B 5/4058; A61B 5/4094; A61B 5/4836; A61B 5/6868; A61B 5/7264; A61B 5/742; A61B 5/746; A61B 5/031

(57) ABSTRACT

A method for detecting a change in a neurological condition in a subject is provided herein. The method includes delivering electrical stimulation to the subject’s brain, measuring a cortical reverberation response to the electrical stimulation, analyzing characteristics of the measured cortical reverberation response, and determining a likelihood of change in the neurological condition in the subject based on changes in the analyzed characteristics of the cortical reverberation response over time. The electrical stimulation may include low-intensity electrical pulses delivered at regular intervals. The analyzed characteristics may include amplitude, duration, or decay slope of the cortical reverberation response. A progressive decline in these characteristics over time may indicate autoregulatory failure or other neurological condition changes in the subject.

### CROSS-REFERENCE TO RELATED APPLICATIONS

This application claims the benefit of U.S. Provisional Patent Application Serial No. 63/793,283, filed on Apr. 23, 2025, and entitled "CORTICAL REVERBERATION MONITORING SYSTEM FOR NEUROLOGICAL ASSESSMENT," which is herein incorporated by reference in its entirety.

### BACKGROUND

Traumatic brain injury (TBI) remains a leading cause of disability and mortality worldwide, presenting significant challenges in clinical management and patient care. Current monitoring techniques for TBI patients often rely on passive measurements of electroencephalography (EEG) signals and physiological parameters, such as intracranial pressure (ICP). These methods, while valuable, may not always provide early detection of neurological deterioration or offer real-time insights into the functional state of the brain.

Neurocritical care units face ongoing difficulties in accurately assessing and predicting secondary injury processes in TBI patients. Existing monitoring approaches may not fully capture the dynamic nature of brain function or detect subtle changes that precede clinical manifestations of worsening injury. There is a growing interest in developing more proactive and sensitive methods for evaluating brain health in acute neurological conditions, potentially enabling earlier interventions and improved patient outcomes.

### SUMMARY OF THE DISCLOSURE

According to an aspect of the present disclosure, a method for detecting a change in a neurological condition in a subject is provided. The method includes delivering electrical stimulation to the subject’s brain, measuring a cortical reverberation response to the electrical stimulation, analyzing characteristics of the measured cortical reverberation response, and determining a likelihood of change in the neurological condition in the subject based on changes in the analyzed characteristics of the cortical reverberation response over time.

According to another aspect of the present disclosure, a method for comprehensive neurological monitoring is provided. The method includes delivering electrical stimulation to a subject’s brain tissue, measuring a cortical reverberation response to the electrical stimulation, monitoring intracranial pressure of the subject, detecting seizure activity in the subject, monitoring for cortical spreading depolarization in the subject, and integrating data from the cortical reverberation response, intracranial pressure, seizure activity, and cortical spreading depolarization to assess the subject’s neurological status.

According to another aspect of the present disclosure, a system for cortical reverberation monitoring is provided. The system includes electrodes to deliver electrical stimulation to a subject’s brain and to measure cortical responses to the delivered electrical stimulation, a processor to analyze characteristics of the measured cortical reverberation responses, and a controller to adjust stimulation parameters of the electrical stimulation delivered by the electrodes based on the analyzed characteristics of the cortical reverberation responses.

### BRIEF DESCRIPTION OF THE DRAWINGS

FIG. 1 is a block diagram of an example neuromodulation system configured as a cortical reverberation monitoring system according to some aspects of the present disclosure.

FIG. 2 is a series of graphs illustrating phase-dependent cortical reverberation responses, showing how evoked response characteristics may vary based on the phase of ongoing brain oscillations at the time of stimulation.

FIG. 3 is a flowchart of an example method for evoking, monitoring, and analyzing cortical reverberations to assess a neurological condition of a subject.

FIGS. 4A and 4B illustrate a temporal relationship between cortical reverberation decline and intracranial pressure elevation, showing how cortical reverberation responses may degrade hours before clinically significant intracranial pressure elevations occur.

FIG. 5 is a block diagram of an example cortical reverberation monitoring system according to some aspects of the present disclosure.

FIG. 6 is a block diagram of example components that can implement the system of FIG. 5.

### DETAILED DESCRIPTION

Described here are systems and methods for monitoring and assessing neurological function using cortical reverberation. This technology may implement a closed-loop neuromodulation system designed to provide real-time assessment of brain states in conditions such as traumatic brain injury (TBI), epilepsy, stroke, headache, migraine, and neuropsychiatric disorders. Additionally or alternatively, other neurological conditions can also be assessed, such as neural implications of tinnitus, subarachnoid hemorrhage, and hypoxic brain injury. Unlike traditional monitoring approaches that passively record neural activity, the disclosed cortical reverberation monitoring systems employ an active perturbation technique, utilizing precisely timed electrical stimulation to query brain function and extract meaningful physiological biomarkers. Advantageously, the electrical stimulation waveform properties are engineered to generate reverberatory neural responses that reflect the underlying dynamics of brain activity, metabolic state, and pathological changes.

As used herein, the term “cortical reverberation” may refer to the brain’s ability to sustain and propagate neural activity following a controlled electrical stimulation or other perturbation. In a healthy brain, the response is strong and sustained. In a brain that is beginning to fail, this response weakens. Cortical reverberation may be measured by delivering low-amplitude electrical pulses to probe cortical function and analyzing the resulting evoked responses. The characteristics of these responses, such as amplitude, duration, and decay pattern, may provide information about the underlying dynamics of brain activity, metabolic state, and pathological changes.

By analyzing how the brain responds to controlled electrical pulses, the systems and methods described herein may provide insights into brain health and function. As a non-limiting example, declining cortical reverberation may signal impending autoregulatory failure or other neurological deterioration before clinical symptoms appear. Cortical reverberation measurements may be obtained using electrodes placed on or within the brain tissue, and may be analyzed in real-time to provide a functional biomarker of brain health.

In some cases, the cortical reverberation monitoring systems described in the present disclosure may include electrodes for delivering electrical stimulation to the brain, electrodes for recording the brain’s response to such electrical stimulation, or electrodes that both deliver electrical stimulation and record the brain’s response thereto. The system may analyze characteristics of the evoked neural activity, such as amplitude, duration, and decay rate, to assess brain function. This active probing approach may allow for dynamic evaluation of the brain’s state.

Current clinical methods for brain monitoring—including intracranial pressure (ICP) sensors, cerebral blood flow monitors, and electroencephalography (EEG)—are all passive measurement techniques with inherent limitations, such as low signal-to-noise ratio (SNR) and susceptibility to external artifacts. The cortical reverberation monitoring system overcomes these limitations by leveraging cortical reverberation as a functional biomarker, in which the brain’s response to stimulation provides real-time insights into neural health.

The cortical reverberation monitoring system has applications in various neurological conditions. For example, the system may be used to detect early signs of deterioration in TBI patients before conventional monitoring methods. This has several advantages: it allows for early detection of worsening autoregulatory failure in TBI, identification of seizure onset in epilepsy, monitoring of cortical spreading depolarizations (CSDs) in stroke, and assessment of network dysfunction in other neurological and/or neuropsychiatric diseases. By actively engaging with the brain, the cortical reverberation monitoring system can detect subtle changes in neural excitability and connectivity that would otherwise be missed by passive monitoring.

By providing an active method to query brain function, the cortical reverberation monitoring system may offer advantages over passive recording techniques. The system may enable earlier detection of pathological changes and more precise characterization of brain states. This information may assist clinicians in making timely treatment decisions and personalizing patient care.

Advantageously, the cortical reverberation monitoring system can be seamlessly integrated into existing clinical workflows. The electrode interface of the system can be placed via standard neurosurgical procedures without requiring additional modifications to surgical protocols. Furthermore, because the system can operate in the background without interrupting conventional brain monitoring, it enhances current ICU and neurology practices rather than replacing them. In addition to invasive applications, the system has the potential to be adapted for non-invasive use, leveraging transcranial or scalp-based stimulation techniques to enable broader accessibility in outpatient or emergency settings.

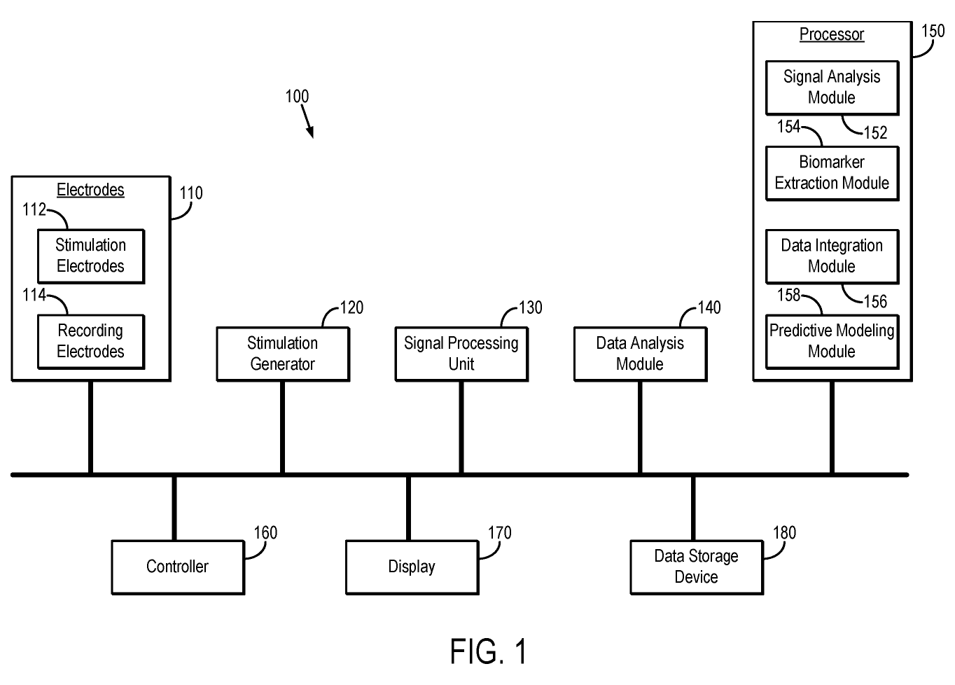

Referring now to FIG. 1, an example cortical reverberation monitoring system 100 capable of active probing and analysis of brain function is illustrated. The cortical reverberation monitoring system 100 may include an electrodes 110, which may include stimulation electrodes 112 and/or recording electrodes 114; a stimulation generator 120; a signal processing unit 130; and a data analysis module 140. In some cases, the signal processing unit 130 and/or data analysis module 140 may be implemented by a processor 150. The cortical reverberation monitoring system 100 may also include a controller 160 to control functions of the electrodes 110, the stimulation generator 120, and/or other components of the cortical reverberation monitoring system 100.

The electrodes 110 may include invasive electrodes, non-invasive electrodes, or both, to deliver electrical pulses and record neural responses. In some cases, the electrodes 110 may incorporate deep brain stimulation (DBS) electrodes for invasive stimulation along with scalp EEG electrodes for non-invasive recording. This multi-modal approach may allow for targeted stimulation of deep brain structures while simultaneously capturing widespread cortical activity.

The stimulation electrodes 112 may be configured to deliver controlled electrical pulses to the brain tissue. In some implementations, the stimulation electrodes 112 may be stereo-electroencephalography (SEEG) electrodes, which can be implanted deep within the brain parenchyma. The SEEG electrodes may have multiple contacts along their length, allowing for stimulation at various depths and locations. In other cases, subdural strip or grid electrodes placed on the cortical surface may be used for stimulation.

In some implementations, electrodes may be placed in the dorsolateral prefrontal cortex (DLPFC), a region that may be routinely accessed for intracranial pressure monitoring and that may reliably yield high-quality evoked responses with minimal risk. Electrode placement may be coordinated with neurosurgical procedures to ensure proximity to clinical monitoring hardware while preserving patient safety. This placement approach may allow for integration of cortical reverberation monitoring into existing clinical workflows without requiring additional modifications to surgical protocols.

The recording electrodes 114 may be utilized to capture the brain’s response to the electrical stimulation. These recording electrodes 114 may be the same electrodes used for stimulation, operating in a different mode, or they may be separate dedicated recording electrodes 114. In some implementations, high-density electrode arrays with up to 256 channels may be employed to provide detailed spatial mapping of cortical responses.

The stimulation generator 120 may be a programmable device capable of producing precisely timed and shaped electrical pulses. In some cases, the stimulation generator 120 can deliver biphasic, charge-balanced pulses with adjustable amplitude, duration, and frequency. The stimulation generator 120 may be capable of producing pulses with amplitudes ranging from 0.1 to 10 mA and durations from 50 to 500 microseconds. In certain implementations, the stimulation generator 120 may support more complex stimulation patterns, such as burst stimulation or theta-burst protocols.

A signal processing unit 130 may be included to amplify, filter, and digitize the recorded neural signals. In some cases, the signal processing unit 130 may incorporate high-precision analog-to-digital converters with sampling rates up to 30 kHz per channel to capture fast neural events. The signal processing unit 130 may additionally or alternatively include hardware-based filters to remove stimulation artifacts and common-mode noise. The signal processing unit 130 may also perform real-time signal conditioning, such as notch filtering to remove power line interference.

In some implementations, the signal processing unit 130 may average cortical reverberation features into defined time bins, such as five-minute bins, to reduce noise and facilitate analysis. The features averaged may include peak amplitude, response duration, decay slope, and waveform morphology. These averaged features may be synchronized with ongoing physiological and clinical data, such as intracranial pressure measurements, seizure logs, and cortical spreading depolarization events, to enable comprehensive assessment of brain health over time.

The cortical reverberation monitoring system 100 may also integrate impedance and signal quality monitoring into its real-time analysis. This integration may ensure that clinicians receive reliable, high-quality data by providing feedback on electrode functionality and signal integrity. In some cases, the system may alert clinicians when signal quality falls below acceptable thresholds or when electrode impedance changes indicate potential issues with the recording interface.

The data analysis module 140 may implement specialized software algorithms running on dedicated computing hardware, or may alternatively be implemented by the processor 150. The data analysis module 140 may perform various analyses on the recorded signals, including time-domain and frequency-domain analyses, connectivity measures, and machine learning-based classification of brain states and/or neurological conditions. In some implementations, the data analysis module 140 may employ adaptive algorithms that determine adjusted stimulation parameters based on the detected cortical responses, enabling closed-loop operation of the system via the controller 160.

A processor 150 may be used to analyze the digitized cortical reverberation responses. The processor 150 may extract relevant features from the evoked potentials, such as response amplitude, latency, and decay characteristics. In these instances, the processor 150 may implement the signal processing unit 130 and/or data analysis module 140. Additionally or alternatively, the processor 150 may implement additional modules, units, or functionalities, including a signal analysis module 152, a biomarker extraction module 154, a data integration module 156, and/or a predictive modeling module 158. In some implementations, the processor 150 may employ machine learning algorithms to detect subtle changes in the cortical reverberation patterns over time.

A controller 160 may be included to manage the overall operation of the cortical reverberation monitoring system 100. The controller 160 may coordinate the timing between stimulation delivery via the stimulation generator 120 and stimulation electrodes 112 and response recording via the recording electrodes 114. Additionally, the controller 160 may adjust stimulation parameters based on the analyzed cortical reverberation data to optimize monitoring sensitivity.

In certain cases, the system may include additional components such as a display 170 to display a user interface for clinicians to monitor real-time brain function and adjust system parameters as needed, and a data storage device 180 for long-term archiving of recorded signals and analysis results. For example, a user interface may be implemented by the processor 150 and the display 170 to display the processed data and analysis results to clinicians. In some cases, this interface may provide real-time visualizations of cortical reverberation metrics, trend analyses, and alerts for significant changes in brain state. A data storage device 180 (e.g., a memory or other suitable data storage device or media) may continuously log all raw signals, processed data, and analysis results. This stored information may be used for retrospective analysis, system refinement, or integration with electronic health records.

In some implementations, the cortical reverberation monitoring system 100 may incorporate data compression and storage methods that enable long-term monitoring while minimizing computational overhead. These methods may allow for continuous archiving of cortical reverberation data over extended monitoring periods, such as days to weeks, without overwhelming storage capacity or processing resources. The compressed data may be stored locally on the data storage device 180 or transmitted to remote servers for cloud-based analysis and long-term archiving.

The cortical reverberation monitoring system 100 may be designed to seamlessly integrate with existing neuromonitoring equipment commonly used in clinical settings. In some cases, the system may be compatible with standard ICP monitors, EEG systems, and other neurophysiological monitoring devices.

In some implementations, the cortical reverberation system 100 may interface with ICP monitoring equipment to correlate changes in cortical reverberation with fluctuations in intracranial pressure. This integration may allow clinicians to observe potential relationships between declining cortical reverberation and rising ICP, potentially providing earlier detection of impending autoregulatory failure.

The cortical reverberation monitoring system 100 may also be designed to work in conjunction with conventional EEG monitoring systems. In some cases, the cortical stimulation and recording components (e.g., stimulation electrodes 112 and/or recording electrodes 114) of the cortical reverberation system 100 may utilize existing EEG electrode placements, minimizing the need for additional invasive procedures. The cortical reverberation monitoring system 100 may process and display cortical reverberation data alongside standard EEG waveforms, allowing for comprehensive neurophysiological assessment.

In some implementations, the cortical reverberation monitoring system 100 may include data integration capabilities to combine its outputs with information from other monitoring devices, such as cerebral blood flow monitors, brain tissue oxygen sensors, and/or microdialysis systems. This multi-modal approach may provide a more comprehensive view of brain health and function in critical care settings.

Advantageously, the cortical reverberation system 100 may be designed with standardized data output formats compatible with existing ICU central monitoring stations, which can increase its ease of use. This may allow cortical reverberation data to be displayed alongside other vital signs and neurological parameters on existing screens and interfaces familiar to clinical staff.

In some cases, a cloud-based neuromonitoring platform may be developed to enable remote patient tracking and clinical decision support. This platform may securely aggregate data from the cortical reverberation system 100 along with other integrated monitoring devices. The cloud-based system may allow authorized clinicians to access real-time patient data, trend analysis, and automated alerts from any location with internet access. The remote monitoring capabilities of the cloud-based platform may facilitate telemedicine applications in neurocritical care. In some implementations, the cloud-based system may enable remote specialists to review cortical reverberation data and other integrated neuromonitoring parameters to provide consultations or guide treatment decisions for patients in facilities without on-site neurocritical care expertise. The cloud-based platform may also incorporate machine learning algorithms to analyze trends in cortical reverberation data alongside other physiological parameters. These algorithms may be designed to identify patterns predictive of neurological deterioration, potentially allowing for earlier intervention and improved patient outcomes.

In some cases, the integration of the cortical reverberation system with existing monitoring equipment and the development of a cloud-based platform may enable the creation of a comprehensive neuromonitoring ecosystem. This ecosystem may provide clinicians with a holistic view of brain function and health, combining active cortical assessment through reverberation measurements with passive monitoring of various physiological parameters.

The cortical reverberation monitoring system 100 may be used to assess brain function in real-time. For example, the cortical reverberation monitoring system 100 may be used to evoke, monitor, and analyze cortical reverberations to assess one or more neurological conditions in a brain of a subject. In some cases, the system may begin by delivering low-intensity electrical stimulation pulses to the brain tissue via electrodes (e.g., stimulation electrodes 112). These pulses may be generated by the stimulation generator 120 and transmitted through leads to the electrodes (e.g., stimulation electrodes 112).

A stimulation protocol may be controlled by a central processing unit (e.g., processor 150) and/or controller 160, which may determine the timing, amplitude, and waveform characteristics of the pulses. In some implementations, the stimulation parameters may be adaptively adjusted based on feedback from the recorded neural responses. For example, following each stimulation pulse, the cortical reverberation monitoring system 100 may record the evoked neural activity using the electrodes 110, such as by using the same electrodes as those used for delivering the electrical stimulation or using different electrodes (e.g., recording electrodes 114). The recorded signals may be amplified and digitized by a data acquisition module implemented by the processor 150. In some cases, this module may also perform initial filtering to remove stimulation artifacts and improve signal quality.

The digitized neural signals may then be processed by a signal analysis module 152 implemented by the processor 150. This module may extract relevant features from the evoked responses, such as amplitude, latency, and waveform shape. In some implementations, advanced signal processing techniques, including time-frequency analysis or machine learning algorithms, may be applied to characterize the cortical reverberation.

A biomarker extraction module 154 implemented by the processor 150 may analyze the processed signals to compute metrics related to cortical reverberation. These metrics may include measures of neural excitability, network connectivity, or the brain’s ability to sustain and propagate activity. In some cases, the cortical reverberation monitoring system 100 may compare current measurements to baseline values or predefined thresholds to detect changes in neurological condition or brain state.

The cortical reverberation monitoring system 100 may incorporate a data integration module 156 (e.g., implemented by processor 150) that combines cortical reverberation measurements with other physiological data, such as intracranial pressure, cerebral blood flow, or EEG readings. As an example, the EEG reading may be used to detect seizure activity in the subject and/or to monitor for cortical spreading depolarization in the subject. This integrated approach may provide a more comprehensive assessment of brain health.

In some implementations, a predictive modeling module 158 may use the collected data to forecast potential changes in brain state, neurological condition, or the risk of neurological deterioration. This predictive modeling may involve machine learning algorithms trained on historical patient data and known clinical outcomes.

In some implementations, the cortical reverberation monitoring system 100 may feature a closed-loop control module implemented by the controller 160 that automatically adjusts stimulation parameters or triggers predefined interventions based on detected changes in cortical reverberation. This adaptive functionality may allow for more responsive and personalized patient monitoring.

The various modules and components of the system may communicate through a central data bus or network, ensuring synchronized operation and rapid information exchange. In some cases, the system may also include wireless communication capabilities for remote monitoring or data transmission to cloud-based analysis platforms.

Throughout its operation, the cortical reverberation monitoring system 100 may employ error checking and fault detection mechanisms to ensure data integrity and system reliability. In some implementations, redundant components or backup power sources may be incorporated to maintain continuous monitoring in critical care settings.

The cortical reverberation monitoring system 100 may be designed to operate continuously over extended periods, with minimal need for manual intervention. However, the cortical reverberation monitoring system 100 may allow for manual override and parameter adjustment by authorized medical personnel to accommodate specific clinical needs or research protocols.

As a non-limiting example, the stimulation protocol used to evoke cortical reverberations may be customized based on the specific application and brain region being targeted. In some cases, the pulse amplitude may range from 0.5 to 5 milliamps, with pulse widths between 60 and 450 microseconds. The stimulation frequency may vary from low-frequency pulses around 1 Hz up to high-frequency bursts of 100-200 Hz.

Different stimulation patterns may be employed for various purposes. For example, a low-frequency stimulation protocol of 1 Hz may be used to probe cortical excitability and assess reverberation, while higher frequency patterns in the gamma range (30-100 Hz) may be applied to modulate neural networks. In some cases, theta-burst stimulation including short bursts of high-frequency pulses delivered at a theta rhythm (5 Hz) may be used to induce plasticity.

As a non-limiting example, the stimulation may be delivered once every three seconds using brief, biphasic, charge-balanced pulses. This three-second interval may be selected because it provides a window to detect seizures between pulses, minimizes the energy delivered to the subject, and provides a sufficient number of pulses to average over five-minute bins for sensitive data analysis. Other stimulation intervals, such as intervals ranging from one to ten seconds, may also be employed depending on the specific clinical application and patient condition.

The cortical reverberation monitoring system 100 may also implement additional neuromodulation techniques. In some cases, transcranial magnetic stimulation (TMS) may be used to deliver focal stimulation to superficial cortical areas. Alternatively, transcranial direct current stimulation (tDCS) or transcranial alternating current stimulation (tACS) may be applied via scalp electrodes to modulate broader regions of cortex. These non-invasive methods may allow for stimulation protocols to be applied in outpatient settings or in cases where invasive electrodes are not feasible.

The stimulation protocol may be adaptive, with parameters adjusted in real-time based on the recorded neural responses as described above. For instance, the stimulation amplitude or frequency may be automatically increased if the evoked response falls below a certain threshold. This closed-loop approach may enable more precise and personalized neuromodulation.

In some implementations, the stimulation may be phase-locked to ongoing brain rhythms detected in the EEG. For example, stimulation pulses may be timed to coincide with specific phases of theta or alpha oscillations to enhance or disrupt particular neural processes.

The duration of stimulation may vary depending on the application. Brief single pulses or short trains lasting milliseconds to seconds may be used for probing cortical excitability. Longer stimulation periods of several minutes may be employed for inducing lasting changes in neural plasticity. In some cases, intermittent stimulation protocols with alternating ON and OFF periods may be used to prevent neural adaptation.

Safety considerations may be incorporated into the stimulation protocol. Charge-balanced biphasic pulses may be used to minimize tissue damage. Limits on the total charge delivered and the duration of continuous stimulation may be enforced. The system may also include automatic safety shutoffs if certain physiological parameters exceed predefined thresholds.

The signal processing and analysis of cortical reverberation data may involve several techniques to extract meaningful information about brain states or other neurological conditions. In some cases, machine learning algorithms may be used for automated detection of pathological brain states and/or neurological conditions based on cortical reverberation data. These algorithms may analyze features such as the amplitude, duration, and decay characteristics of evoked responses to identify patterns indicative of autoregulatory failure, seizure activity, cortical spreading depolarizations, or other neurological conditions or changes in neurological conditions.

In some implementations, the cortical reverberation monitoring system 100 may employ supervised learning approaches, where the machine learning algorithms are trained on labeled datasets of cortical reverberation responses associated with known brain states or neurological conditions. The trained models may then be applied to new data to classify brain states in real-time. Unsupervised learning techniques may also be utilized to discover novel patterns or clusters in the cortical reverberation data that may correspond to different physiological or pathological conditions.

Feature extraction methods may be applied to the raw cortical reverberation signals to reduce dimensionality and highlight relevant characteristics. These features may include time-domain measures such as peak amplitude and latency, frequency-domain measures derived from spectral analysis, and wavelet-based time-frequency representations. The extracted features may serve as inputs to the machine learning models for brain state classification.

Cortical reverberation analysis may also be used to measure phase/amplitude dependency and phase-dependent plasticity in the brain. Phase/amplitude coupling, which refers to the relationship between the phase of low-frequency oscillations and the amplitude of high-frequency activity, may be quantified using metrics such as the modulation index or phase-locking value. These measures may provide insights into the functional connectivity and information flow within neural networks.

To assess phase-dependent plasticity, the cortical reverberation monitoring system 100 may analyze how the cortical reverberation response changes based on the phase of ongoing brain oscillations at the time of stimulation. This analysis may involve binning the evoked responses according to the phase of a specific frequency band (e.g., theta or alpha) and comparing the response characteristics across different phase bins. Statistical techniques such as circular statistics may be employed to determine if there are significant phase-dependent effects on the evoked response.

Data visualization techniques may be used to represent the complex multidimensional data derived from cortical reverberation analysis. These may include heat maps to display phase-amplitude coupling across different frequency pairs, polar plots to show phase-dependent effects on evoked responses, and time-frequency spectrograms to visualize dynamic changes in the frequency content of the signals over time.

In some cases, the cortical reverberation monitoring system 100 may implement adaptive thresholding algorithms to account for inter-individual variability and changes in baseline brain activity over time. These algorithms may continuously update the criteria for detecting abnormal cortical reverberation patterns based on recent historical data from the individual patient.

The signal processing pipeline may also incorporate artifact rejection methods to minimize the impact of stimulation artifacts, movement-related noise, and other sources of interference on the cortical reverberation analysis. These methods may include template subtraction, independent component analysis, or machine learning-based approaches for identifying and removing artifactual components of the signal.

By combining these various signal processing and analysis techniques, the system may provide a comprehensive assessment of brain function based on cortical reverberation data, enabling early detection of pathological states and insights into neural plasticity mechanisms.

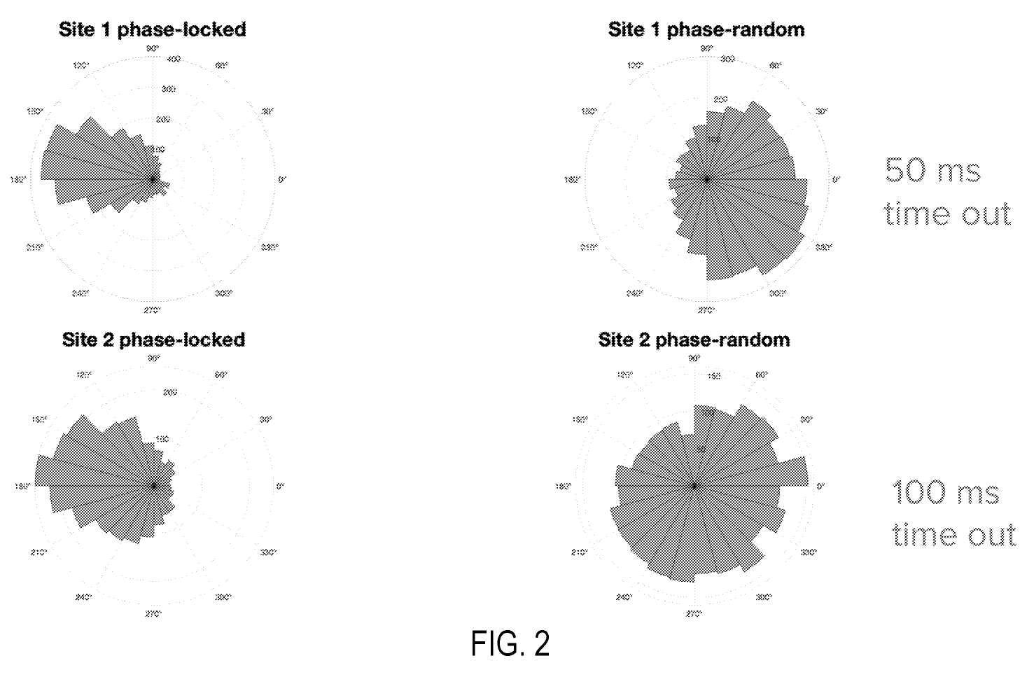

Referring now to FIG. 2, a series of graphs illustrating phase-dependent cortical reverberation responses is shown. Preliminary data has shown statistical changes in cortico-cortical evoked potential (CCEP) characteristics when comparing phase-locked versus random-phase stimulation. In some implementations, phase preference in evoked responses may reveal natural rhythm bands in a perturbative manner. The system may extract phase information from recorded signals using techniques such as wavelet decomposition across multiple frequency bands, for example from approximately 3 Hz to 30 Hz. Evoked responses may be aligned to stimulation time and sorted by phase to analyze phase-dependent effects on response amplitude and latency. In some cases, these phase-dependent variations in cortical reverberation may provide additional insights into brain state and neural excitability.

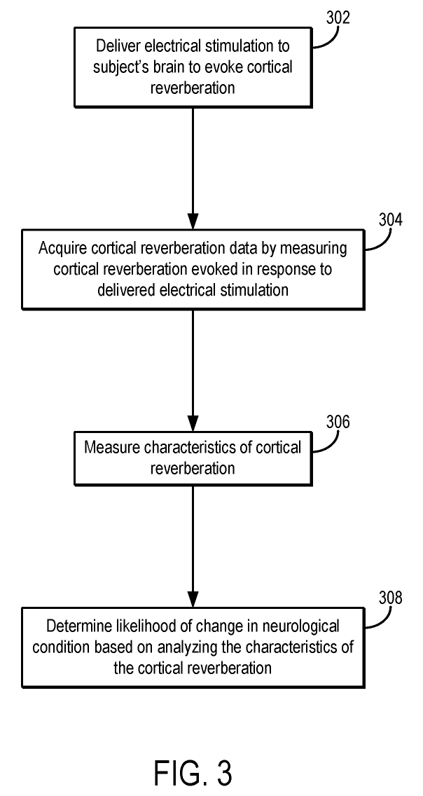

Referring now to FIG. 3, a flowchart is illustrated as setting forth the steps of an example method for evoking, monitoring, and analyzing cortical reverberation in a patient to assess a neurological condition of the patient. This method enables real-time assessment of brain function by actively probing neural tissue, providing a more sensitive and dynamic measure of neurological status compared to passive monitoring techniques.

The method includes delivering electrical stimulation to the subject’s brain, as indicated at step 302. The electrical stimulation may include low-intensity electrical pulses delivered at regular intervals. Delivering low-intensity pulses at regular intervals allows for continuous monitoring of brain function while minimizing the risk of inducing seizures or other adverse effects. The electrical stimulation may be delivered at frequencies ranging from 0.1 Hz to 100 Hz to probe different aspects of neural function. In some implementations, the electrical stimulation may be delivered at regular intervals, such as once every three seconds. This stimulation timing may provide a window to detect seizures between pulses while minimizing the energy delivered to the subject. Additionally, this interval may provide a sufficient number of pulses to average over defined time periods, such as five-minute bins, to enable sensitive data analysis. Other stimulation intervals may also be used depending on the specific clinical application and monitoring requirements.

A cortical reverberation response to the electrical stimulation is measured, as indicated at step 304. The cortical reverberation response may be measured using intracranial electrodes, scalp electrodes, or a combination of both to provide comprehensive spatial coverage of brain activity.

Characteristics of the measured cortical reverberation response are analyzed, as indicated at step 306. Analyzing characteristics of the measured cortical reverberation response may include evaluating at least one of amplitude, duration, or decay slope of the cortical reverberation response. Evaluating multiple characteristics of the cortical reverberation response provides a comprehensive assessment of brain function, capturing different aspects of neural excitability and connectivity.

A likelihood of change in the neurological condition in the subject is determined based on changes in the analyzed characteristics of the cortical reverberation response over time, as indicated at step 308. Determining the likelihood of the change in the neurological condition in the subject may include detecting a progressive decline in at least one of the amplitude, duration, or decay slope of the cortical reverberation response over time. Detecting progressive declines in cortical reverberation characteristics enables early identification of deteriorating brain function, potentially before clinical symptoms manifest. In some cases, the change in the neurological condition in the subject may include autoregulatory failure in the subject. Identifying autoregulatory failure early allows for timely interventions to prevent secondary brain injury and improve patient outcomes.

In some embodiments, the method may further include comparing the analyzed characteristics of the cortical reverberation response to baseline measurements obtained from the subject. Comparing responses to patient-specific baselines accounts for individual variability in brain function and enhances the sensitivity of the monitoring technique.

In some other embodiments, the method may further include generating an alert when the analyzed characteristics deviate from the baseline measurements by a predetermined threshold. Automated alert generation ensures timely notification of clinicians when significant changes in brain function are detected, facilitating rapid clinical response.

The cortical reverberation monitoring system 100 may have applications across various neurological conditions beyond traumatic brain injury. In some cases, the cortical reverberation monitoring system 100 may be used to monitor and assess brain function in epilepsy patients. For epilepsy monitoring, the cortical reverberation technique may provide insights into seizure susceptibility and excitability. The system may detect changes in evoked responses that precede seizure onset, potentially allowing for earlier intervention.

In some cases, the cortical reverberation system may be applied to stroke patients. The system may be used to monitor for CSDs, which are associated with secondary injury progression in stroke. By way of example, a CSD may refer to a wave of near-complete depolarization of neurons and glial cells that propagates across the cerebral cortex. This phenomenon is characterized by a dramatic shift in cellular membrane potentials, accompanied by significant changes in extracellular ion concentrations, particularly potassium. CSDs may be associated with a temporary cessation of spontaneous and evoked neuronal activity, followed by a period of depressed electrocorticographic activity. In some aspects, CSD may occur in response to various insults to the brain, including TBI, ischemia, hemorrhage, or hypoxia. The propagation speed of CSD may typically range from 2 to 5 millimeters per minute. This event may be accompanied by changes in cerebral blood flow, metabolic alterations, and potential tissue swelling. Detection and monitoring of CSDs may provide important insights into the evolution of brain injury and may serve as a biomarker for ongoing or impending neurological deterioration in conditions such as TBI, stroke, and subarachnoid hemorrhage. By actively probing cortical function, the system may detect CSDs with greater sensitivity than passive monitoring methods.

In some implementations, CSDs may be detected by the presence of slow potential shifts greater than 500 milliseconds in duration propagating across adjacent cortical electrodes, often followed by transient suppression of spontaneous activity. These detection criteria may be implemented in a semi-automated analysis pipeline, with identified events undergoing manual validation by an experienced reviewer. By comparing cortical reverberation patterns before, during, and after CSD events, the system may determine whether cortical reverberation degrades in advance of CSDs, potentially serving as an early biomarker of evolving neurophysiological instability.

For seizure detection, the cortical reverberation monitoring system 100 may identify seizures based on the sudden onset of rhythmic, high-amplitude discharges with evolving frequency. In some cases, detected seizure events may be confirmed by expert neurophysiologists. The system may compare cortical reverberation characteristics in patients with and without seizures to assess whether changes in cortical reverberation precede or coincide with seizure activity.

In some implementations, electrodes may be placed in specific brain regions that reliably yield high-quality evoked responses. As a non-limiting example, electrodes may be placed in the dorsolateral prefrontal cortex (DLPFC), a region that may be routinely accessed for intracranial pressure monitoring and that may provide robust cortical reverberation measurements with minimal risk. Other brain regions may also be targeted depending on the specific clinical application and patient anatomy.

The cortical reverberation monitoring system 100 may assess how changes in cortical reverberation relate to impending clinical deterioration using defined thresholds. As non-limiting examples, such thresholds may include sustained intracranial pressure elevation greater than 23 mmHg, escalation in medical therapy such as hyperosmolar agents or hyperventilation, or the need for surgical intervention such as decompressive craniectomy. In some cases, computational modeling has demonstrated that cortical reverberation declines may be detectable in more than 80% of cases prior to intracranial pressure elevation, and that cortical reverberation responses may degrade hours before clinically significant intracranial pressure elevations occur.

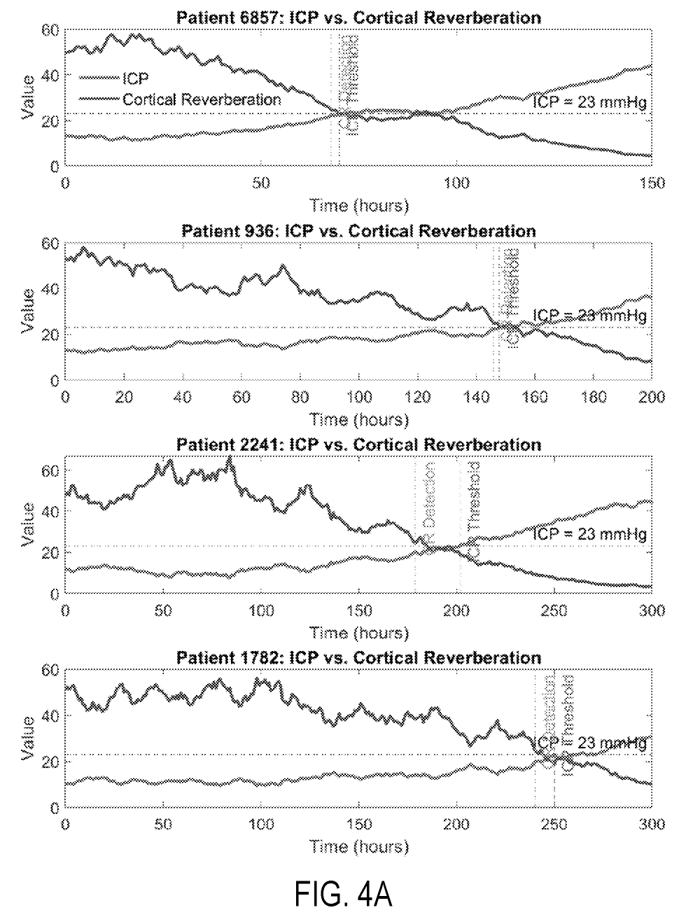

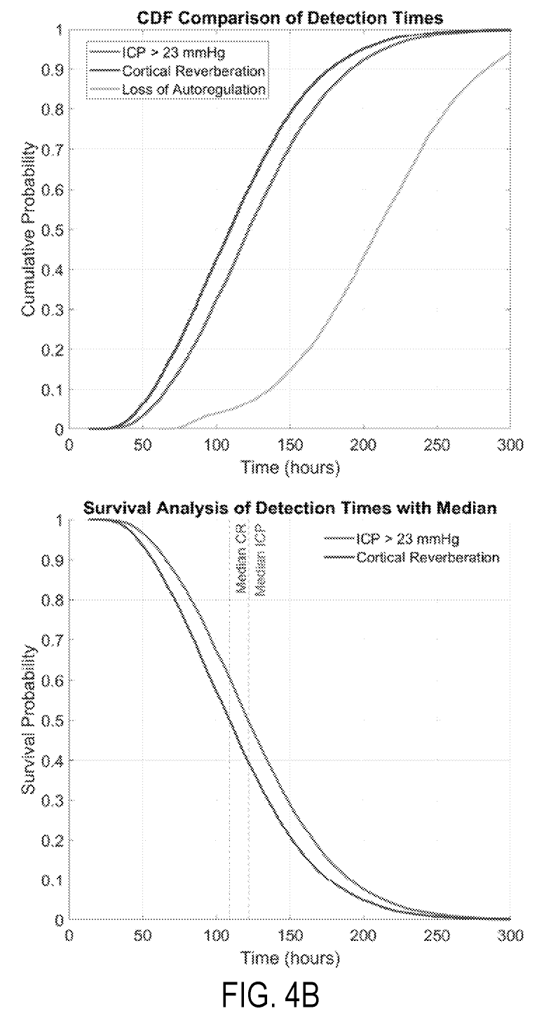

Referring now to FIGS. 4A and 4B, graphs illustrating the temporal relationship between cortical reverberation decline and intracranial pressure elevation are shown. In some implementations, preliminary data may suggest that cortical reverberation responses degrade hours before clinically significant ICP elevations occur. Computational modeling simulating patients with worsening TBI has demonstrated that monitoring cortical reverberation may provide additional time for clinical decision-making compared to conventional ICP monitoring alone. In some cases, this temporal relationship suggests that cortical reverberation may serve as an early warning biomarker for impending autoregulatory failure, potentially giving clinicians more time to act to preserve brain tissue and function.

The cortical reverberation technique may also have applications in neuropsychiatric disorders. In some cases, the system may be used to assess network dysfunction and connectivity changes in conditions such as depression or schizophrenia. The evoked responses measured by the system may provide quantitative biomarkers of brain network alterations in these disorders.

In addition to detecting autoregulatory failure, seizures, and CSDs, the cortical reverberation system may be used to map connections in the brain. By stimulating at one location and measuring responses at multiple other sites, the cortical reverberation monitoring system 100 may generate functional connectivity maps. This mapping capability may be useful for both research and clinical applications, such as pre-surgical planning for epilepsy patients.

The cortical reverberation monitoring system 100 may also be adapted for use in pharmaceutical research and drug development. In some cases, the cortical reverberation monitoring system 100 may be used to evaluate the effects of neurological drugs and neuroprotective agents on brain function. The quantitative measures of cortical excitability and connectivity provided by the cortical reverberation monitoring system 100 may serve as biomarkers for assessing drug efficacy in clinical trials. For example, the cortical reverberation monitoring system 100 may be used to measure changes in evoked responses before and after administration of an experimental neuroprotective agent in TBI patients.

In some cases, the cortical reverberation system 100 may be applied in combination with other neuromodulation therapies. For instance, the system may be used to assess the effects of deep brain stimulation or transcranial magnetic stimulation on cortical network dynamics as described above. The evoked responses measured by the cortical reverberation monitoring system 100 may provide feedback on the efficacy of these interventions and guide parameter optimization.

In some cases, the cortical reverberation monitoring system 100 may be used to measure plasticity after neuromodulation treatment. By comparing cortical reverberation responses before and after therapeutic interventions such as deep brain stimulation, transcranial magnetic stimulation, or other neuromodulation therapies, the system may provide quantitative assessment of treatment-induced changes in neural excitability and connectivity. This application may enable clinicians to evaluate the efficacy of neuromodulation treatments and guide parameter optimization for individual patients.

The versatility of the cortical reverberation monitoring system 100 may allow for its application across a wide range of clinical scenarios. By providing active, quantitative assessment of brain function, the system may enhance diagnostic capabilities, guide treatment decisions, and advance understanding of various neurological and psychiatric conditions.

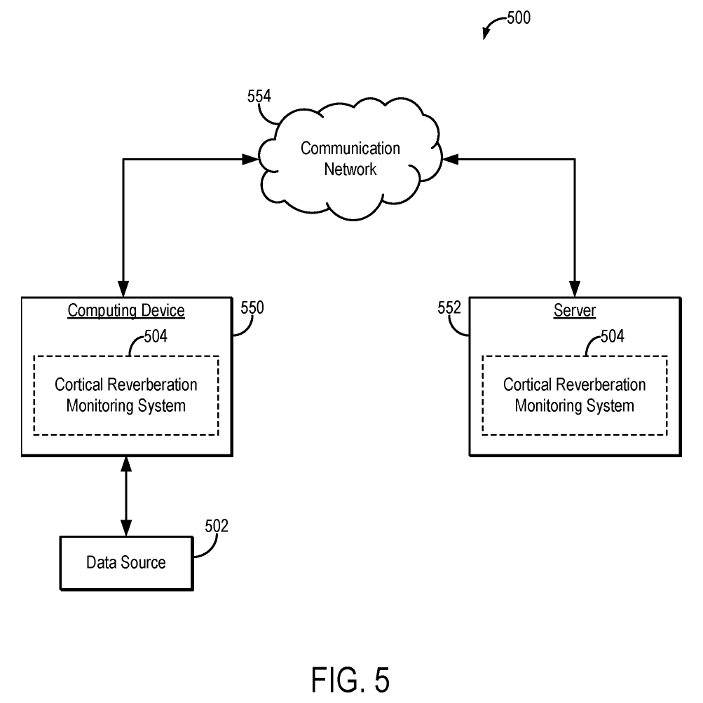

FIG. 5 shows an example of a system 500 for evoking, monitoring, and analyzing cortical reverberation data in accordance with some embodiments described in the present disclosure. As shown in FIG. 5, a computing device 550 can receive one or more types of data (e.g., cortical reverberation data, EEG data, other physiological data) from data source 502. In some embodiments, computing device 550 can execute at least a portion of a cortical reverberation monitoring system 504 to monitor and analyze cortical reverberation data received from the data source 502.

Additionally or alternatively, in some embodiments, the computing device 550 can communicate information about data received from the data source 502 to a server 552 over a communication network 554, which can execute at least a portion of the cortical reverberation monitoring system 504. In such embodiments, the server 552 can return information to the computing device 550 (and/or any other suitable computing device) indicative of an output of the cortical reverberation monitoring system 504.

In some embodiments, computing device 550 and/or server 552 can be any suitable computing device or combination of devices, such as a desktop computer, a laptop computer, a smartphone, a tablet computer, a wearable computer, a server computer, a virtual machine being executed by a physical computing device, and so on.

In some embodiments, data source 502 can be any suitable source of data (e.g., measurement data, processed measurement data), such as recording electrodes, another computing device (e.g., a server storing measurement data, processed measurement data), and so on. In some embodiments, data source 502 can be local to computing device 550. For example, data source 502 can be incorporated with computing device 550 (e.g., computing device 550 can be configured as part of a device for measuring, recording, estimating, acquiring, or otherwise collecting or storing data). As another example, data source 502 can be connected to computing device 550 by a cable, a direct wireless link, and so on. Additionally or alternatively, in some embodiments, data source 502 can be located locally and/or remotely from computing device 550, and can communicate data to computing device 550 (and/or server 552) via a communication network (e.g., communication network 554).

In some embodiments, communication network 554 can be any suitable communication network or combination of communication networks. For example, communication network 554 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some embodiments, communication network 554 can be a local area network, a wide area network, a public network (e.g., the Internet), a private or semi-private network (e.g., a corporate or university intranet), any other suitable type of network, or any suitable combination of networks. Communications links shown in FIG. 5 can each be any suitable communications link or combination of communications links, such as wired links, fiber optic links, Wi-Fi links, Bluetooth links, cellular links, and so on.

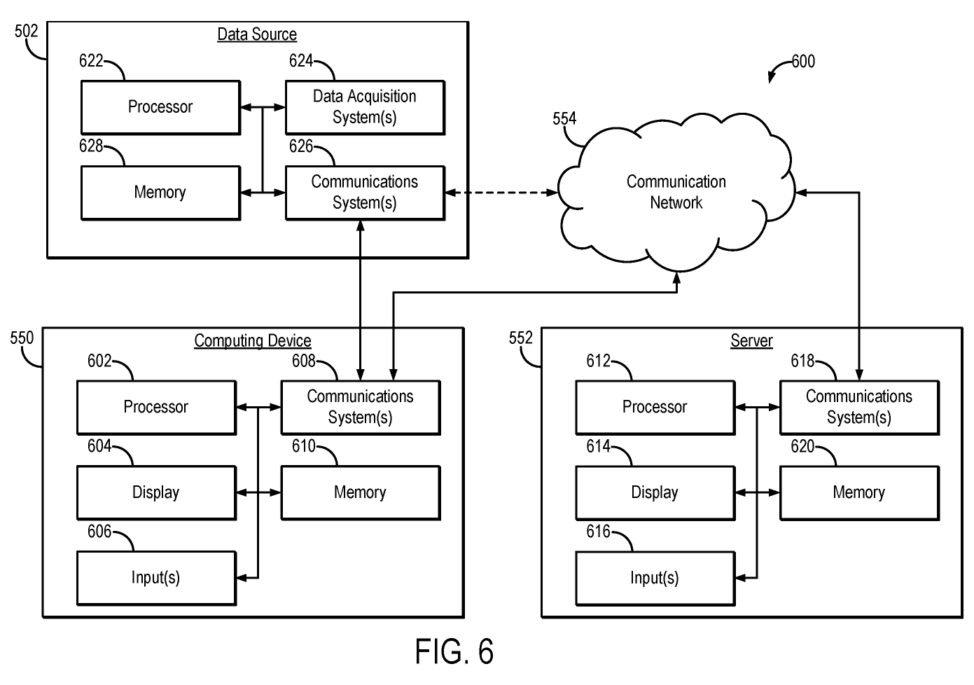

Referring now to FIG. 6, an example of hardware 600 that can be used to implement data source 502, computing device 550, and server 552 in accordance with some embodiments of the systems and methods described in the present disclosure is shown.

As shown in FIG. 6, in some embodiments, computing device 550 can include a processor 602, a display 604, one or more inputs 606, one or more communication systems 608, and/or memory 610. In some embodiments, processor 602 can be any suitable hardware processor or combination of processors, such as a central processing unit (CPU), a graphics processing unit (GPU), and so on. In some embodiments, display 604 can include any suitable display devices, such as a liquid crystal display (LCD) screen, a light-emitting diode (LED) display, an organic LED (OLED) display, an electrophoretic display (e.g., an “e-ink” display), a computer monitor, a touchscreen, a television, and so on. In some embodiments, inputs 606 can include any suitable input devices and/or sensors that can be used to receive user input, such as a keyboard, a mouse, a touchscreen, a microphone, and so on.

In some embodiments, communications systems 608 can include any suitable hardware, firmware, and/or software for communicating information over communication network 554 and/or any other suitable communication networks. For example, communications systems 608 can include one or more transceivers, one or more communication chips and/or chip sets, and so on. In a more particular example, communications systems 608 can include hardware, firmware, and/or software that can be used to establish a Wi-Fi connection, a Bluetooth connection, a cellular connection, an Ethernet connection, and so on.

In some embodiments, memory 610 can include any suitable storage device or devices that can be used to store instructions, values, data, or the like, that can be used, for example, by processor 602 to present content using display 604, to communicate with server 552 via communications system(s) 608, and so on. Memory 610 can include any suitable volatile memory, non-volatile memory, storage, or any suitable combination thereof. For example, memory 610 can include random-access memory (RAM), read-only memory (ROM), electrically programmable ROM (EPROM), electrically erasable ROM (EEPROM), other forms of volatile memory, other forms of non-volatile memory, one or more forms of semi-volatile memory, one or more flash drives, one or more hard disks, one or more solid state drives, one or more optical drives, and so on. In some embodiments, memory 610 can have encoded thereon, or otherwise stored therein, a computer program for controlling operation of computing device 550. In such embodiments, processor 602 can execute at least a portion of the computer program to present content (e.g., images, user interfaces, graphics, tables), receive content from server 552, transmit information to server 552, and so on. For example, the processor 602 and the memory 610 can be configured to perform the methods described herein.

In some embodiments, server 552 can include a processor 612, a display 614, one or more inputs 616, one or more communications systems 618, and/or memory 620. In some embodiments, processor 612 can be any suitable hardware processor or combination of processors, such as a CPU, a GPU, and so on. In some embodiments, display 614 can include any suitable display devices, such as an LCD screen, LED display, OLED display, electrophoretic display, a computer monitor, a touchscreen, a television, and so on. In some embodiments, inputs 616 can include any suitable input devices and/or sensors that can be used to receive user input, such as a keyboard, a mouse, a touchscreen, a microphone, and so on.

In some embodiments, communications systems 618 can include any suitable hardware, firmware, and/or software for communicating information over communication network 554 and/or any other suitable communication networks. For example, communications systems 618 can include one or more transceivers, one or more communication chips and/or chip sets, and so on. In a more particular example, communications systems 618 can include hardware, firmware, and/or software that can be used to establish a Wi-Fi connection, a Bluetooth connection, a cellular connection, an Ethernet connection, and so on.

In some embodiments, memory 620 can include any suitable storage device or devices that can be used to store instructions, values, data, or the like, that can be used, for example, by processor 612 to present content using display 614, to communicate with one or more computing devices 550, and so on. Memory 620 can include any suitable volatile memory, non-volatile memory, storage, or any suitable combination thereof. For example, memory 620 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 620 can have encoded thereon a server program for controlling operation of server 552. In such embodiments, processor 612 can execute at least a portion of the server program to transmit information and/or content (e.g., data, images, a user interface) to one or more computing devices 550, receive information and/or content from one or more computing devices 550, receive instructions from one or more devices (e.g., a personal computer, a laptop computer, a tablet computer, a smartphone), and so on.

In some embodiments, the server 552 is configured to perform the methods described in the present disclosure. For example, the processor 612 and memory 620 can be configured to perform the methods described herein.

In some embodiments, data source 502 can include a processor 622, one or more data acquisition systems 624, one or more communications systems 626, and/or memory 628. In some embodiments, processor 622 can be any suitable hardware processor or combination of processors, such as a CPU, a GPU, and so on. In some embodiments, the one or more data acquisition systems 624 are generally configured to acquire neural response data (e.g., cortical reverberation data) and can include recording electrodes, an electrode array, or the like. Additionally or alternatively, in some embodiments, the one or more data acquisition systems 624 can include any suitable hardware, firmware, and/or software for coupling to and/or controlling operations of recording electrodes, an electrode array, or the like. In some embodiments, one or more portions of the data acquisition system(s) 624 can be removable and/or replaceable.

Note that, although not shown, data source 502 can include any suitable inputs and/or outputs. For example, data source 502 can include input devices and/or sensors that can be used to receive user input, such as a keyboard, a mouse, a touchscreen, a microphone, a trackpad, a trackball, and so on. As another example, data source 502 can include any suitable display devices, such as an LCD screen, an LED display, an OLED display, an electrophoretic display, a computer monitor, a touchscreen, a television, etc., one or more speakers, and so on.

In some embodiments, communications systems 626 can include any suitable hardware, firmware, and/or software for communicating information to computing device 550 (and, in some embodiments, over communication network 554 and/or any other suitable communication networks). For example, communications systems 626 can include one or more transceivers, one or more communication chips and/or chip sets, and so on. In a more particular example, communications systems 626 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 628 can include any suitable storage device or devices that can be used to store instructions, values, data, or the like, that can be used, for example, by processor 622 to control the one or more data acquisition systems 624, and/or receive data from the one or more data acquisition systems 624; to generate images from data; present content (e.g., data, images, a user interface) using a display; communicate with one or more computing devices 550; and so on. Memory 628 can include any suitable volatile memory, non-volatile memory, storage, or any suitable combination thereof. For example, memory 628 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 628 can have encoded thereon, or otherwise stored therein, a program for controlling operation of data source 502. In such embodiments, processor 622 can execute at least a portion of the program to generate images, transmit information and/or content (e.g., data, images, a user interface) to one or more computing devices 550, receive information and/or content from one or more computing devices 550, receive instructions from one or more devices (e.g., a personal computer, a laptop computer, a tablet computer, a smartphone, etc.), and so on.

In some embodiments, any suitable computer-readable media can be used for storing instructions for performing the functions and/or processes described herein. For example, in some embodiments, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

The present disclosure has described one or more preferred embodiments, and it should be appreciated that many equivalents, alternatives, variations, and modifications, aside from those expressly stated, are possible and within the scope of the invention.

1. A method for detecting a change in a neurological condition in a subject, comprising:
delivering electrical stimulation to the subject’s brain;
measuring a cortical reverberation response to the electrical stimulation;
analyzing characteristics of the measured cortical reverberation response; and
determining a likelihood of change in the neurological condition in the subject based on changes in the analyzed characteristics of the cortical reverberation response over time.

2. The method of claim 1, wherein the electrical stimulation comprises low-intensity electrical pulses delivered at regular intervals.

3. The method of claim 1, wherein analyzing characteristics of the measured cortical reverberation response comprises evaluating at least one of amplitude, duration, or decay slope of the cortical reverberation response.

4. The method of claim 3, wherein determining the likelihood of the change in the neurological condition in the subject comprises detecting a progressive decline in at least one of the amplitude, duration, or decay slope of the cortical reverberation response over time.

5. The method of claim 4, wherein the change in the neurological condition in the subject comprises autoregulatory failure in the subject.

6. The method of claim 1, further comprising comparing the analyzed characteristics of the cortical reverberation response to baseline measurements obtained from the subject.

7. The method of claim 6, further comprising generating an alert when the analyzed characteristics deviate from the baseline measurements by a predetermined threshold.

8. A method for comprehensive neurological monitoring, comprising:
delivering electrical stimulation to a subject’s brain tissue;
measuring a cortical reverberation response to the electrical stimulation;
monitoring intracranial pressure of the subject;
detecting seizure activity in the subject;
monitoring for cortical spreading depolarization in the subject; and
integrating data from the cortical reverberation response, intracranial pressure, seizure activity, and cortical spreading depolarization to assess the subject’s neurological status.

9. The method of claim 8, wherein delivering electrical stimulation comprises applying low-intensity electrical pulses at regular intervals to the subject’s brain tissue.

10. The method of claim 9, wherein the regular intervals are between 1 and 10 seconds.

11. The method of claim 8, wherein measuring the cortical reverberation response comprises analyzing at least one of amplitude, duration, or decay slope of the response.

12. The method of claim 11, further comprising detecting a progressive decline in at least one of the amplitude, duration, or decay slope of the cortical reverberation response over time to indicate neurological deterioration.

13. The method of claim 8, further comprising generating an alert when the integrated data indicates a predetermined level of neurological deterioration.

14. The method of claim 13, wherein the alert triggers an automated adjustment of at least one treatment parameter for the subject.

15. A system for cortical reverberation monitoring, comprising:
electrodes to deliver electrical stimulation to a subject’s brain and to measure cortical responses to the delivered electrical stimulation;
a processor to analyze characteristics of the measured cortical reverberation responses; and
a controller to adjust stimulation parameters of the electrical stimulation delivered by the electrodes based on the analyzed characteristics of the cortical reverberation responses.

16. The system of claim 15, wherein the electrodes comprise a plurality of stereo-electroencephalography (SEEG) electrodes configured for implantation in the subject’s brain tissue.

17. The system of claim 15, wherein the processor further analyzes at least one of amplitude, duration, or decay slope of the measured cortical reverberation responses.

18. The system of claim 17, wherein the controller adjusts at least one of stimulation frequency, amplitude, or pulse width of the electrical stimulation delivered by the electrode array based on changes in the analyzed characteristics of the cortical reverberation responses.

19. The system of claim 18, further comprising a display to present real-time visualizations of the analyzed cortical reverberation responses and adjusted stimulation parameters.
